(19) United States
(12) Patent Application Publication
(10) Pub. No.: US 2008/0175801 A1
(43) Pub. Date: Jul. 24, 2008

(54) Stable peroxide containing personal care compositions

(75) Inventor: Niranjan Ramji, City Mason, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/654,837

(22) Filed: Jan. 18, 2007

(51) Int. Cl. A61K 8/22

(52) U.S. Cl. 424/53

(57) ABSTRACT

The present invention relates to stable personal care composition, including oral care compositions containing a peroxide source. The compositions are stabilized by eliminating or minimizing the presence in the composition of metals having radical forming potential with the peroxide. Preferably, the metals that are eliminated or reduced are cobalt, copper, palladium, nickel and iron. The compositions are further stabilized by the addition of agents having scavenging or quenching activity for free radicals. Reducing free radical activity in the product matrix prevents radical-mediated loss and degradation of peroxide and other ingredients, in particular organic compounds added as active or aesthetic agents, including flavors, perfumes, colorants and thickeners. Provided are peroxide containing oral care products with enhanced consumer appeal in terms of taste, mouthfeel and appearance, thereby encouraging compliance and regular use. Such attributes are important since use of these products may involve fairly long residence time in the mouth for efficacy.

### FIELD OF THE INVENTION

The present invention relates to stable personal care composition, including oral care compositions containing a peroxide source. The compositions are stabilized against loss and degradation of peroxide and other composition ingredients, in particular organic compounds added as active or aesthetic agents, including flavors, perfumes, colorants and thickeners. Peroxide sources are included in personal care compositions such as dentifrice and mouthrinse for bleaching/whitening and antimicrobial benefits. However formulating stable peroxide containing compositions presents significant challenges for various reasons mainly arising from the reactivity of peroxide and the instability or susceptibility to degradation of many composition ingredients in the presence of peroxide or reactive species derived from peroxide such as free radicals. Encompassed in the present invention are peroxide-containing oral care products stabilized by reducing free radical activity in the product matrix and thus against radical-mediated loss and degradation of peroxide and organic compound components including oral care actives, flavorants, colorants and other aesthetic ingredients. Provided are peroxide containing products with enhanced consumer appeal in terms of taste, mouthfeel and appearance, thereby encouraging compliance and regular use. Such attributes are important since use of these products may involve fairly long residence time in the mouth for efficacy.

### BACKGROUND OF THE INVENTION

Oral care products such as dentifrice and mouthrinse are routinely used by consumers as part of their oral care hygiene regimens. It is well known that oral care products can provide both therapeutic and cosmetic hygiene benefits to consumers. Therapeutic benefits include caries prevention which is typically delivered through the use of various fluoride salts; gingivitis prevention by the use of an antimicrobial agent such as triclosan, stannous fluoride, essential oils; or cetylpyridinium chloride or hypersensitivity control through the use of ingredients such as strontium chloride or potassium nitrate. Cosmetic benefits provided by oral care products include the control of plaque and calculus formation, removal and prevention of tooth stain, tooth whitening, breath freshening, and overall improvements in mouth feel impression which can be broadly characterized as mouth feel aesthetics. Calculus and plaque, along with behavioral and environmental factors, lead to formation of dental stains, significantly affecting the aesthetic appearance of teeth. Behavioral and environmental factors that contribute to teeth staining propensity include regular use of coffee, tea, cola or tobacco products, and also the use of certain oral products containing ingredients that promote staining, such as cationic antimicrobials and metal salts.

Thus daily oral care at home requires products with multiple ingredients working by different mechanisms to provide the complete range of therapeutic and aesthetic benefits, including anticaries, antimicrobial, antigingivitis, antiplaque and anticalculus as well as anti-odor, mouth refreshment, stain removal, stain control and tooth whitening. In order for oral care products for daily use such as dentifrice and rinses to provide complete oral care it is necessary to combine actives and additives, many of which have the disadvantage of causing negative aesthetics during use, in particular unpleasant taste and sensations and stain promotion. The unpleasant taste and mouth sensations have been described as having one or more of bitter, metallic, astringent, salty, numbing, stinging, burning, prickling, and even irritating aspects. Typical ingredients for oral care use that are associated with these aesthetic negatives include antimicrobial agents such as cetyl pyridinium chloride, chlorhexidine, stannous and zinc salts; tooth bleaching agents such as peroxides; antitartar agents such as pyrophosphate, tripolyphosphate and hexametaphosphate; and excipients such as baking soda and surfactants. To mitigate the aesthetic negatives from these ingredients, oral care products are typically formulated with flavoring agents and sweeteners to taste as good as possible and be consumer acceptable.

Although there have been many advances in oral care product formulations in recent years, there is still a need for improved products, particularly peroxide containing products with improved stability, aesthetics and taste. Formulating peroxide containing products presents significant challenges mainly because many traditional organic compound components including actives, flavorants, colorants and other aesthetic ingredients are not sufficiently stable in the presence of peroxide and peroxide-derived reactive species, especially free radicals.

### SUMMARY OF THE INVENTION

The present invention provides peroxide-containing compositions that are stabilized against metal-mediated degradation of peroxide via the Fenton reaction, which results in the formation of hydroxyl free radicals that cause product instability including loss and degradation of peroxide itself and many organic compound components, change in product rheology, as well as or degradation and loss of integrity of packaging materials. The present peroxide-containing compositions are stabilized by eliminating or minimizing the presence in the composition of metals having radical forming potential with the peroxide. The compositions are formulated to be essentially free of these metals, meaning that the concentration in the composition of such metals is reduced to zero or no more than a specified limit for each metal of 1.8 ppb Chromium (Cr), 0.6 ppb Manganese (Mn), 9 ppb Iron (Fe), 0.07 ppb Cobalt (Co), 10 ppb Nickel (Ni), 1 ppb Copper (Cu), 0.3 ppb Molybdnenum (Mo), 0.09 ppb Palladium (Pd), 0.06 ppb Silver (Ag), and 0.045 ppb Platinum(Pt). Preferably, the metals that are eliminated or reduced are cobalt, copper, palladium, nickel and iron. The compositions are further stabilized by the addition of agents having scavenging or quenching activity for free radicals. By “stabilized” herein is meant that free radical activity in the product is substantially eliminated or significantly reduced such that the product does not undergo unacceptable loss or degradation of peroxide and other formulation ingredients, in particular organic compounds functioning as actives, flavorant, fragrance, colorant, rheology agent, and package material. Thus, the product retains its physical and chemical properties for extended periods of time, due to significant reduction in the rate of degradation of formulation components compared to unstabilized formulations. In the present compositions, the peroxide component retains most of its initial activity as oxidant and antimicrobial; the active components retain most of their potency and activity and the flavors, perfumes, colorants and rheology agents retain their ability to impart desired aesthetics to the composition.

These and other features, aspects, and advantages of the invention will become evident to those skilled in the art from a reading of the present disclosure.

### DETAILED DESCRIPTION OF THE INVENTION

While the specification concludes with claims particularly pointing out and distinctly claiming the invention, it is believed that the present invention will be better understood from the following description.

All percentages and ratios used hereinafter are by weight of total composition, unless otherwise indicated. All percentages, ratios, and levels of ingredients referred to herein are based on the actual amount of the ingredient, and do not include solvents, fillers, or other materials with which the ingredient may be combined as a commercially available product, unless otherwise indicated.

All measurements referred to herein are made at 25° C. unless otherwise specified.

Herein, “comprising” means that other steps and other components which do not affect the end result can be added. This term encompasses the terms “consisting of” and “consisting essentially of.”

As used herein, the word “include,” and its variants, are intended to be non-limiting, such that recitation of items in a list is not to the exclusion of other like items that may also be useful in the materials, compositions, devices, and methods of this invention.

As used herein, the words “preferred”, “preferably” and variants refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

By “oral care composition” or “oral composition” is meant a product, which in the ordinary course of usage, is not intentionally swallowed for purposes of systemic administration of particular therapeutic agents, but is rather retained in the oral cavity for a time sufficient to contact substantially all of the dental surfaces and/or oral tissues for purposes of oral activity. In addition to cleaning teeth to remove dental plaque, oral care compositions function to prevent the formation of dental calculus and disorders such as caries, periodontitis and gingivitis, and also to eliminate and prevent oral malodor or halitosis and staining. Examples of oral care product forms include toothpaste, dentifrice, tooth gel, subgingival gel, mouthrinse, mouthspray, mousse, foam, denture product, lozenge, chewable tablet or chewing gum and strips or films for direct application or attachment to oral surfaces.

The term “dentifrice”, as used herein, means paste, gel, or liquid formulations unless otherwise specified. The dentifrice composition may be a single phase composition or may be a combination of two or more separate dentifrice compositions. The dentifrice composition may be in any desired form, such as deep striped, surface striped, multilayered, having the gel surrounding the paste, or any combination thereof. Each dentifrice composition in a dentifrice comprising two or more separate dentifrice compositions may be contained in a physically separated compartment of a dispenser and dispensed side-by-side.

The term “dispenser”, as used herein, means any pump, tube, or container suitable for dispensing compositions such as dentifrices and mouthrinses.

The term “teeth”, as used herein, refers to natural teeth as well as artificial teeth or dental prosthesis.

Herein, the terms “tartar” and “calculus” are used interchangeably and refer to mineralized dental plaque biofilms.

The term “orally acceptable carrier” as used herein includes any safe and effective materials for use in the compositions of the present invention. Such materials include conventional additives in oral care compositions including but not limited to fluoride ion sources, anti-calculus or anti-tartar agents, desensitizing agents, other teeth whitening agents, abrasives such as silica, herbal agents, chelating agents, buffers, anti-staining agents, alkali metal bicarbonate salts, thickening materials, humectants, water, surfactants, titanium dioxide, flavor system, sweetening agents, xylitol, coloring agents, and mixtures thereof.

Active and other ingredients useful herein may be categorized or described herein by their cosmetic and/or therapeutic benefit or their postulated mode of action or function. However, it is to be understood that the active and other ingredients useful herein can, in some instances, provide more than one cosmetic and/or therapeutic benefit or function or operate via more than one mode of action. Therefore, classifications herein are made for the sake of convenience and are not intended to limit an ingredient to the particularly stated application(s).

The essential and optional ingredients of the present compositions are described in the following paragraphs.

### Peroxide Source

The present compositions contain a peroxide source as an essential ingredient. Oral care compositions include a source of peroxide for its many benefits to the oral cavity. It has long been recognized that hydrogen peroxide and other peroxygen-containing agents are effective in curative and/or prophylactic treatments with respect to caries, dental plaque, gingivitis, periodontitis, mouth odor, tooth stains, recurrent aphthous ulcers, denture irritations, orthodontic appliance lesions, postextraction and postperiodontal surgery, traumatic oral lesions and mucosal infections, herpetic stomatitis and the like. Peroxide-containing agents in the oral cavity exert a chemomechanical action generating thousands of tiny oxygen bubbles produced by interaction with tissue and salivary enzymes. The swishing action of a mouthrinse enhances this inherent chemomechanical action. Such action has been recommended for delivery of other agents into infected gingival crevices. Peroxide mouthrinses prevent colonization and multiplication of anaerobic bacteria known to be associated with periodontal disease. However, compositions containing hydrogen peroxide or other peroxide releasing compounds generally are difficult to formulate for stability reasons. Peroxides or reactive species derived from peroxide interact with other common excipients in the composition and tend to be unstable in storage, continuously losing the capacity to release active or nascent oxygen over relatively short periods of time, and diminishing or destroying the desired function of formulation excipients. Among such excipients are flavorants, sensory materials and coloring agents which are added to enhance the acceptability of the oral care product.

Hydogen peroxide is a preferred peroxide source. It is well established that hydrogen peroxide decomposes by the exothermic decomposition to oxygen and water. The reactions which occur with peroxide which impact peroxide and formulation stability are a) decomposition b) oxidation and c) reduction. Pure hydrogen peroxide is relatively stable, but the problems occur when hydrogen peroxide is contaminated, or contain impurities. High quality (90%) H₂O₂ has a decomposition rate of <0.0010%/hr (<9%/yr) at about 50° C. Stabilizers are thus added to supplies of hydrogen peroxide to reduce the decomposition rate to tolerable levels. If all impurities or contamination can be removed from hydrogen peroxide then it is theoretically possible to have a product that will lose less than 0.5% of its strength at 30° C. over 12 months. However, in reality this is very difficult to achieve. For example, there is unavoidable leaching of metals from containers used during the making of peroxide itself or during the making of the finished product. Transition metal contaminants are known to catalyze peroxide degradation.

In addition to reactivity with “impurities” such as metals, other factors which influence the stability of peroxide include temperature and pH. An increase in temperature results in greater decomposition of peroxide. In general, higher pH results in greater decomposition of peroxide. The decomposition at high pH proceeds as shown below.

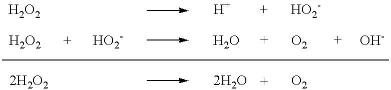

Decomposition of peroxide also occurs by reaction with any compound or element that has a potential to get oxidized or reduced. For example, oxidation of organic compounds during bleaching reactions results in the reduction of peroxide to water. Or reduction of oxidants can result in peroxide oxidation. Elements or complexes that can assume multiple valence states, transition metals in particular, are very good decomposition catalysts. The decomposition rate is dependent upon the species of transition metal. Cyclic redox decomposition can also occur if there are species such as catalysts which can get oxidized and reduced as illustrated in the following reactions.

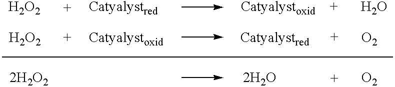

The majority of the decomposition routes for peroxide occur under alkaline conditions and it is for that reason that peroxide containing products are generally formulated under acidic or mildly acidic conditions. However even under acidic conditions, the metal-mediated free radical decomposition of peroxide (referred to as Fenton reaction) is a predominant pathway and results in considerable formulation instability. The catalytic decomposition of peroxide by the Fenton mechanism is catalyzed primarily by heavy metals, especially transition metals, which starts by formation of hydroxyl free radicals. The type of metal, its oxidation state, whether chelated or colloidal, and the pH of solution are factors that can affect the degree of the Fenton reaction. Many commonly used formulation excipients especially polymeric materials contain trace metal contamination that can trigger such metal catalyzed generation of hydroxyl free radicals. The hydroxyl free radical is a very reactive species generation of hydroxyl free radicals. The hydroxyl free radical is a very reactive species and free radical reactions are self-propagating becoming a chain reaction that continues until a termination product is produced. By such time, in the absence of any stabilization means, both the peroxide and many organic components could be destroyed. Once formed, free radicals are free to combine with many organic species in the composition. Such free radicals would be especially reactive with compounds having conjugated double bonds, for example, dyes, colorants and many flavor and perfume chemicals.

Metal catalyzed free radical decomposition of peroxide under acidic conditions by the Fenton mechanism generally involve transition metals, in particular Chromium (Cr), Manganese (Mn), Iron (Fe), Cobalt (Co), Nickel (Ni), Copper (Cu), Molybdnenum (Mo), Palladium (Pd), Silver (Ag), and Platinum(Pt). Fenton reactions involving a transition metal such as iron (Fe) are illustrated below.

### Primary Reactions:

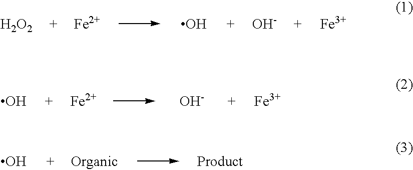

### Secondary Reactions:

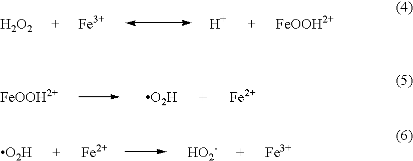

In the Fenton reaction of divalent metal with peroxide, reaction (1) is the rapid rate determining step in which the divalent metal is consumed quickly to generate hydroxyl free radicals but is reproduced slowly as the rate of reaction (4) is much slower than reaction (1).

The relative order of reactivity of transition metals towards peroxide resulting in the formation of free radicals is shown below as Relative Radical Forming Potential (RRFP) values, determined using a chemiluminesence assay described in more detail below. The assay measures the increase in the chemiluminescence signal provided by each metal over a control without the metal. The chemiluminescence signal represents the hydroxyl free radicals that are formed (i.e., % increase over control=the Radical Forming Potential). The assays are conducted over a concentration range of 10 ug/ml to 0.0001 ug/ml of metal. The sum of all the values over the concentration range is defined as the Relative Radical Forming Potential. The higher the RRFP value, the greater the radical forming potential of the metal. The RRFP may also be represented as an index (RRFPI) relative to Co which was determined to have the highest potential and assigned an index of 100.

| | Metal | | | | | | | | | |
|---|---|---|---|---|---|---|---|---|---|---|
| | Co | Cu | Pd | Ni | Fe | Au | Mn | Mo | Cr | Pt |
| RRFP | 503.87 | 340.13 | 231.58 | 188.64 | 149.04 | 57.28 | 13.95 | 7.52 | 8.92 | 6.99 |
| RRFPI | 100 | 67.5 | 45.96 | 37.43 | 29.57 | 11.36 | 2.76 | 1.49 | 1.77 | 1.38 |

The RRFP values above are obtained using a chemiluminescence assay to monitor free radicals, reactive metabolites and hydrogen peroxide. The assay is based on the measurement of chemiluminescence resulting in the oxidation of luminol with hydrogen peroxide [Journal of Pharmacological and Toxicological Methods, 2000, 43, 183-190]. In the absence of free radicals the reaction probably proceeds via the ionization of peroxide to the hydroperoxide anion as the rate determining step. In the presence of transition metal, the hydroxyl free radical generated from peroxide (Fenton reaction) results in the amplification of chemiluminescence intensity. [Journal of Pharmacology and Toxicological Methods, 2000, 44, 507-512]. The monodissociated luminol (LH⁻) reacts with hydroxyl radicals (.OH) at basic pH to form water and diazamiquinone radical (L⁻), which in turn reduces O₂ to superoxide anion (O₂⁻.) and is oxidized to 5-aminohyphenthalazine-1,4-dione (LH₂). The reaction between L⁻ and O²⁻ yields the carbon centered hydroperoxide anion (LOO⁻), which rearranges to a transient endoperoxide, which in turn decomposes to give the light emission and the end products, an aminophthalate (AP) and N₂. In the presence of hydroxyl free radicals the oxidative reaction of luminol to aminophthalate is more facile, thus leading to an enhancement of the chemiluminescence signal intensity. Oxygen centered radicals such as hydroxyl and alkoxyl radicals formed by hemolytic scission of hydroperoxide, also cause photoemissive luminol oxidation.

Luminol gives high chemiluminescence signals at pH 9; however its signal intensity diminishes at pH 7. For evaluations at lower pH values, a derivative of luminol is used, L-012 (8-amino-5-chloro-7-phenylpyrido [3,4-dipyridazine-1,4-(2H,3H)-dione sodium salt), which is a highly sensitive chemiluminescence probe (about 100 times greater signal intensity than luminol). The mechanism of chemiluminescence with L-012 is likely similar to that of luminol.

To evaluate the Relative Radical Forming Potential (RRFP) of test samples such as transition metal compounds, test samples (transition metal sample+buffer+peroxide+luminol or L-012) were run against a blank set (transition metal+buffer+peroxide) and a positive control set (luminol or L-012+buffer+peroxide).

The RRFP values are reported as % increase of chemiluminescence signal vs. control. Using the chemiluminescence assay described above, the permissible levels of metals with radical forming potential in the compositions are established. At these levels, the ability of the metal to mediate free radical generation is eliminated or sufficiently reduced, resulting in stabilization of the composition. The permissible level varies by metal species as follows: 1.8 ppb Chromium (Cr), 0.6 ppb Manganese (Mn), 9 ppb Iron (Fe), 0.07 ppb Cobalt (Co), 10 ppb Nickel (Ni), 1 ppb Copper (Cu), 0.3 ppb Molybdnenum (Mo), 0.09 ppb Palladium (Pd), 0.06 ppb Silver (Ag), and 0.045 ppb Platinum(Pt).

The present peroxide-containing compositions are thus formulated to be essentially free of such metals with significant radical forming potential, meaning that the concentration of such metals are reduced to below the above limits. The compositions may be prepared by subjecting to a metal removal process. Metal removal can be achieved by using cation exchange resins (such as resins supplied by Purolite Corporation or Resin Tech), polymer supported filtration discs (3M Empore High Performance filtration discs), polymer supported chelants (Ethylenediamine modified silica, supplied by Strem Chemicals and Triaminetetracetate silica supported, QuadraPure AMPA from Aldrich Chemical Company). The process involves passing the liquid formulation or excipients through a bed of the above mentioned materials or treating the above mentioned materials with the formulation or excipients in a batch process over a period of time and filtering out the resin/polymer carrying the metals to be removed.

Peroxide sources include peroxide compounds, perborates, percarbonates, peroxyacids, persulfates, and combinations thereof. Suitable peroxide compounds include hydrogen peroxide, urea peroxide, calcium peroxide, sodium peroxide, zinc peroxide and mixtures thereof. A preferred percarbonate is sodium percarbonate. Preferred persulfates include oxones.

Preferred peroxide sources for use in dentifrice formulations include calcium peroxide and urea peroxide. Hydrogen peroxide and urea peroxide are preferred for use in mouthrinse formulations. The following amounts represent the amount of peroxide raw material, although the peroxide source may contain ingredients other than the peroxide raw material. The present composition may contain from about 0.01% to about 30%, preferably from about 0.1% to about 10%, and more preferably from about 0.5% to about 5% of a peroxide source, by weight of the composition.

In addition to a source of peroxide, the present oral care compositions may comprise other components which are described in the following paragraphs.

### Free Radical Scavengers/Quenchers

Further desirable components of the present compositions are additives that function as free radical scavengers and/or quenchers to further reduce free radical activity in the composition. The free radical scavengers work by tying up any free radicals initially formed in the composition. Thus the ability of the free radicals to degrade the organic components is removed at the same time the self-propagating free radical cascade reactions are stopped short. By such a mechanism, degradation of formulation ingredients including actives, flavorants, perfumes, colorants and dyes, surfactants, and thickeners is arrested or greatly reduced. Addition of free radical scavengers to the compositions combined with processing of the compositions and/or ingredients to reduce metal content result in the present highly stable compositions.

Suitable free radical scavengers and/or quenchers include polyphosphates (containing an average number of phosphate units of from 2 to 125, such as tripolyphosphate, n=3 and Glass H polyphosphate, n=21); other phosphate compounds such as monosodium phosphate, calcium phosphate, potassium phosphate, calcium glycerophosphate, manganese hypophosphite and phytates; tin compounds such as sodium stannate, stannous oxide, stannic oxide, stannous chloride, stannous tartrate, stannous fluoride. Other suitable radical scavengers include phenolics (mono- and polyhydroxy benzenes) and derivatives thereof and alkyl- and aryl carboxylates such as described in commonly assigned U.S. Pat. No. 6,001,794. Examples include BHA (butylated hydroxyanisole), BHT (butylated hydroxytoluene), TBHQ (tertiary butyl hydroquinone), propyl gallate, gallic acid (3,4,5-trihydroxybenzoic acid), pyrogallol (1,2,3-trihydroxybenzene), cinnamic acid, caffeic acid (3,4-dihydroxycinnamic acid), coumaric acid, protocatechuic acid (3,4-dihydroxybenzoic acid), o-pyrocatechuic acid (2,3-dihydroxybenzoic acid), α-resorcylic acid (3,5-dihydroxybenzoic acid, β-resorcylic acid (2,4-dihydroxybenzoic acid), benzoic acid, toluic acid, ferulic acid, gallic acid, trans-resveratrol, catechol, t-butyl catechol, 2-methoxy-phenol, 2-ethoxy-phenol, 4-allyl-catechol, 2-methoxy-4-(2-propenyl)phenol.

Additional radical scavengers include flavonoids, isoflavonoids and other phenolics such as quercetin (3,3′,4′,5,7-pentahydroxyflavone), rutin (3,3′,4′,5,7-pentahydroxyflavone-3-rutinoside), morin, kaempferol, fisetin, isorhamnetin, myricetin, catechin, gallocatechin gallate, epicatechin (EC), epigallocatechin (EGC), epigallocatechin gallate (EGCG), epicatechin gallate (ECG), leucocyanidol, oligomeric proanthocyanidins, delphinidin, malvidin, 4-hydroxyphenylacetic acid; polysaccharides such as curdlan (β-1,3-glucan polysaccharide from Alkaligenes faecalis), sodium carboxymethyl betaglucan; vitamins, amino acids and nutrients such as salicylic acid (2-hydroxy benzoic acid), reduced glutathione, uric acid, ascorbic acid, ascorbic acid-2-monophosphate, L-ascorbyl stearate, carnitine (4-N-trimethylammonium-3-hydroxybutyric acid), methionine and folic acid.

Compounds above having free radical scavenging or quenching activity that are derived from natural sources especially plants and herbs, are preferred herein. These natural materials are advantageous in that they have potent radical scavenging activity and are already known to be safe for ingestion. Examples of sources of these materials include oils and extracts of the following: Rosemary (Rosmarinus officinalis), green tea, oak bark, cranberry, Panax ginseng, grape skin, Ginko biloba, St. Johns Wort (Hypericum undulatum); lavender (Lavandula angustifolia); butterfly lavender (Lavandula pedunulata), lemon balm (Melissa officinalis), sage (Salvia officinalis), apple mint (Mentha suaveolens), little burnet (Sanguisorba minor), laurel (Laurus nobilis), Thymus vulgaris, Thymus pulegiodes, Rheum ribes, Globularia alypum L., Origanum majorana L., Melissae folium, Spiraea herba, Uvae ursi folium, Rubi fructose folium, Salicis cortex, Gerani robertiani herba, Serpylli herba, Fragaria herba folium, Hyptis fasciculote, Copernicia speciosa, Orbignya speciosa.

Polyphosphates are also preferred for use herein as free radical scavengers. A polyphosphate is generally understood to consist of two or more phosphate molecules arranged primarily in a linear configuration, although some cyclic derivatives may be present. The inorganic polyphosphate salts desired include pyrophosphate, tripolyphosphate, tetrapolyphosphate and hexametaphosphate, among others. Polyphosphates larger than tetrapolyphosphate usually occur as amorphous glassy materials. Preferred in this invention are the linear polyphosphates having the formula:

XO(XPO₃)ₙX

wherein X is sodium, potassium or ammonium and n averages from about 3 to about 125. Among preferred polyphosphates are those having n averaging from about 3 to about 21 including tripolyphosphate (n=3) and those commercially known as Sodaphos (n=6), Hexaphos (n=13), and Glass H (n=21) and manufactured by FMC Corporation and Astaris. These polyphosphates may be used alone or in combination. The radical scavenging ability is greater, the greater the number of phosphate groups. Thus it is preferred to use longer-chain polyphosphates, such as Glass H polyphosphate with an average chain length of about 21. It is known that polyphosphates are susceptible to hydrolysis in high water formulations at acid pH, particularly below pH 5. It is believed such longer-chain polyphosphates when undergoing hydrolysis produce shorter-chain polyphosphates which still function effectively as radical scavengers. The polyphosphates and other radical scavenging agents may also function to chelate metals, thereby further reducing the availability of these metals to mediate undesirable reactions.

Other phosphorylated compounds may be used in addition to or instead of the polyphosphates, including polyphosphorylated inositol compounds such as phytic acid, myo-inositol pentakis(dihydrogen phosphate); myo-inositol tetrakis(dihydrogen phosphate), myo-inositol trikis(dihydrogen phosphate), and an alkali metal, alkaline earth metal or ammonium salt thereof. Preferred herein is phytic acid, also known as myo-inositol 1,2,3,4,5,6-hexakis (dihydrogen phosphate) or inositol hexaphosphoric acid, and its alkali metal, alkaline earth metal or ammonium salts. Herein, the term “phytate” includes phytic acid and its salts as well as the other polyphosphorylated inositol compounds.

Typically, the compositions herein-comprise at least 0.01% by weight of the total composition of the radical scavenger, or mixtures thereof, preferably from 0.04% to 10%, more preferably from 0.05% to 2.0% and most preferably from 0.05% to 1.0%. Also suitable weight ratio of the peroxide to the radical scavenger or mixtures thereof in the liquid compositions herein is below 500, preferably below 300 and more preferably below 200.

### Flavor System

A flavor system comprising flavoring agents, sweeteners and coolants is typically added to oral care compositions to mitigate aesthetic negatives from ingredients such as peroxide itself and to make the oral care products taste as good as possible and be consumer acceptable. Pleasant tasting compositions improve user compliance to prescribed or recommended use of peroxide containing products. However, most peroxide-containing oral care products in the market suffer from poor consumer appeal because of unpleasant taste or limited flavor choices. Many flavor chemicals (flavorants) are noted for being unstable in the presence of peroxide and reactive species derived from peroxide.

In the present compositions, the flavor system is stabilized against degradation by eliminating or substantially reducing free-radical activity in the compositions and thus, radical-mediated degradation reactions of flavorants. The present invention thus significantly expands the range of flavoring options for peroxide-containing products. By “stabilized” herein is meant degradation of flavor components is significantly reduced, thus minimizing off-flavors and maintaining the flavor character or profile during the life of the product. Examples of flavor and fragrance materials that are effectively stabilized in accordance with the present methods and may be formulated with peroxide include traditional flavorants such as menthol, methyl salicylate, ethyl salicylate, methyl cinnamate, ethyl cinnamate, butyl cinnamate, ethyl butyrate, ethyl acetate, menthyl anthranilate, iso-amyl acetate, iso-amyl butyrate, allyl caproate, eugenol, eucalyptol, thymol, cinnamic alcohol, cinnamic aldehyde, octanol, octanal, decanol, decanal, phenylethyl alcohol, benzyl alcohol, benzaldehyde, alpha-terpineol, linalool, linonene, citral, vanillin, ethyl vanillin, propenyl guaethol, maltol, ethyl maltol, heliotropin, anethole, dihydroanethole, carvone, oxanone, menthone, β-damascenone, ionone, gamma decalactone, gamma nonalactone, gamma undecalactone, 4-hydroxy-2,5-dimethyl-3(2H)-furanone, natural flavor oils or extracts such as peppermint, spearmint, wintergreen, citrus, orange, lime, lemon, other fruits and mixtures thereof. Additional examples of flavor chemicals that may be stabilized in accordance with the present invention are listed below classified according to structural features.

1) Aliphatic saturated aldehydes
Hexanal—occurs in orange and lemon oil
Nonanal—occurs in citrus and rose oil—floral composition
Undecanal—flowery waxy odor, occurs in citrus oil
Dodecanal—waxy odor used in conifer fragrance and citrus note.
Tridecanal—occurs in lemon and cucumber oil
2-Methyldecanal—aldehydic, citrus peel like, waxy-green odor.
2-Methylundecanal—conifer note
Phenylacetaldehyde—hyacinth and rose note
Dihydrocinnamaldehyde—hyacinth and lilac composition
2-phenylpropanal—blossom composition
3-(4-Ethylphenyl)-2,2-dimethylpropanal—fresh air tone reminiscent of ocean breeze
3-(3-Isopropylphenyl)-butanal—floral note
2-methyl-3-(-4-tert-butyl-phenyl-phenyl)-propanal—lily-of-the-valley like odor
   2) Aliphatic unsaturated aldehydes with isolated double bonds
2,6-Dimethyl-5-hepten-1-al—melon and cucumber note
E-4-Decenal—fresh natural citrus like note
10-Undecenal (aiso 9-Undecenal, 8-Undecenal and 7-Undecenal)—fatty green, slightly metallic, heavy flowery odor
2-Dodecenal—orange-mandarin like citrus note
2,6,10-Trimethyl-5,6-undecadienal—fruity
Citronellal—balm mint
4-(4-Methyl-3-penten-1-yl)-3-cyclohene carboxaldehyde—fruity, slightly citrus like
4-(4-Hydroxy-4methylpentyl)-3-cyclohexene carboxaldehyde—lily-of-the-valley like odor
   3) Aliphatic and aromatic hydrocarbons, alcohols, esters and O—, N— and/or S-Heterocycles with isolated double bonds
α- and γ-Terpinene—herbaceous citrus odor
α-Terpinyl acetate—lavender and bergamot type
α-Phellandrene—citrus odor
Isopulegol (8-p-menthen-3-ol)—imparts cooling sensation
α- and β-Pinene—constituent of many volatile oils, e.g., mandarin peel oil
Phenoxyacetic acid allyl ester—green, sweetish, herbal fruity odor
Isoeugenol—clove odor
Isoeugenol methyl ether—mild clove odor
Eugenol methyl ether—mild spicy slightly herbal odor
Eugenol acetate—fruity clove odor
Farnesol—linden blossom odor
Nerolidol—base note in flowery odor
Rose oxide (Isobutenyl Methyltetrahydropyran)—rose and floral fragrance
Linalool oxide—lavender note
   4) Aliphatic conjugated ketones
Nootkatone (4,4α-dimethyl-6-isopropenyl-4,4α,5,6,7,8-hexahydro-3H-napthalen-2-one)—grapefruit odor
Cedryl methyl ketone—long lasting woody fragrance
Cis-Jasmone—jasmine odor
Dihydrojasmone—typical jasmine odor
6,7-Dihydro-1,1,2,3,3-pentamethyl-4(5H)-indanone—conifer like musk odor
3-Methyl-2cyclopenten-2-ol-1-one—caramel note

One or more of these flavorants are generally used in the compositions at levels of from about 0.001% to about 5%, by weight of the composition.

The oral care composition will optionally comprise from about 0.04% to 1.5% coolants such as menthol, menthyl esters and other derivatives, carboxamides, ketals, diols, and mixtures thereof. Examples of coolants useful in the present compositions are the paramenthan carboxamide agents such as N-ethyl-p-menthan-3-carboxamide, known commercially as “WS-3”, N,2,3-trimethyl-2-isopropylbutanamide, known as “WS-23”, and others in the series such as WS-5, WS-11, WS-14 and WS-30. Additional suitable coolants include 3-1-menthoxypropane-1,2-diol known as TK-10 manufactured by Takasago; menthone glycerol acetal known as MGA; menthyl esthers such as menthyl acetate, menthyl acetoacetate, menthyl lactate known as Frescolat® supplied by Haarmann and Reimer, and monomenthyl succinate under the tradename Physcool from V. Mane. The terms menthol and menthyl as used herein include dextro- and levorotatory isomers of these compounds and racemic mixtures thereof. TK-10 is described in U.S. Pat. No. 4,459,425, Amano et al., issued Jul. 10, 1984. WS-3 and other agents are described in U.S. Pat. No. 4,136,163, Watson, et al., issued Jan. 23, 1979.

The flavor system will typically include a sweetening agent. Suitable sweeteners include those well known in the art, including both natural and artificial sweeteners. Some suitable water-soluble sweeteners include monosaccharides, disaccharides and polysaccharides such as xylose, ribose, glucose (dextrose), mannose, galactose, fructose (levulose), sucrose (sugar), maltose, invert sugar (a mixture of fructose and glucose derived from sucrose), partially hydrolyzed starch, corn syrup solids, dihydrochalcones, monellin, steviosides, and glycyrrhizin. Suitable water-soluble artificial sweeteners include soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (acesulfame-K), the free acid form of saccharin, and the like. Other suitable sweeteners include Dipeptide based sweeteners, such as L-aspartic acid derived sweeteners, such as L-aspartyl-L-phenylalanine methyl ester (aspartame) and materials described in U.S. Pat. No. 3,492,131, L-alpha-aspartyl-N-(2,2,4,4-tetramethyl-3-thietanyl)-D-alaninamide hydrate, methyl esters of L-aspartyl-L-phenylglycerin and L-aspartyl-L-2,5,dihydrophenyl-glycine, L-aspartyl-2,5-dihydro-L-phenylalanine, L-aspartyl-L-(1-cyclohexyen)-alanine, and the like. Water-soluble sweeteners derived from naturally occurring water-soluble sweeteners, such as a chlorinated derivative of ordinary sugar (sucrose), known, for example, under the product description of sucralose as well as protein based sweeteners such as thaumatoccous danielli (Thaumatin I and II) can be used. A composition preferably contains from about 0.1% to about 10% of sweetener, preferably from about 0.1% to about 1%, by weight of the composition.

In addition the flavor system may include salivating agents, warming agents, and numbing agents. These agents are present in the compositions at a level of from about 0.001% to about 10%, preferably from about 0.1% to about 1%, by weight of the composition. Suitable salivating agents include Jambu® manufactured by Takasago. Examples of warming agents are capsicum and nicotinate esters, such as benzyl nicotinate. Suitable numbing agents include benzocaine, lidocaine, clove bud oil, and ethanol.

### Other Active Agents

The present compositions may optionally include other active agents, especially antimicrobial agents that provide activity against oral bacterial pathogens and undesirable conditions caused by these pathogens including plaque, gingivitis, periodontal disease and mouth malodor. Included among such agents are water insoluble non-cationic antimicrobial agents such as halogenated diphenyl ethers, phenolic compounds including phenol and its homologs, mono and poly-alkyl and aromatic halophenols, resorcinol and its derivatives, bisphenolic compounds and halogenated salicylanilides, benzoic esters, and halogenated carbanilides. The water soluble antimicrobials include quaternary ammonium salts and bis-biquanide salts, and triclosan monophosphate. The quaternary ammonium agents include those in which one or two of the substitutes on the quaternary nitrogen has a carbon chain length (typically alkyl group) from about 8 to about 20, typically from about 10 to about 18 carbon atoms while the remaining substitutes (typically alkyl or benzyl group) have a lower number of carbon atoms, such as from about 1 to about 7 carbon atoms, typically methyl or ethyl groups. Dodecyl trimethyl ammonium bromide, tetradecylpyridinium chloride, domiphen bromide, N-tetradecyl-4-ethyl pyridinium chloride, dodecyl dimethyl (2-phenoxyethyl) ammonium bromide, benzyl dimethylstearyl ammonium chloride, cetyl pyridinium chloride, quaternized 5-amino-1,3-bis(2-ethyl-hexyl)-5-methyl hexa hydropyrimidine, benzalkonium chloride, benzethonium chloride and methyl benzethonium chloride are exemplary of typical quaternary ammonium antibacterial agents. Other compounds are bis[4-(R-amino)-1-pyridinium] alkanes as disclosed in U.S. Pat. No. 4,206,215, issued Jun. 3, 1980, to Bailey. Other antimicrobials such as copper salts, zinc salts and stannous salts may also be included. Also useful are enzymes, including endoglycosidase, papain, dextranase, mutanase, and mixtures thereof. Such agents are disclosed in U.S. Pat. No. 2,946,725, Jul. 26, 1960, to Norris et al. and in U.S. Pat. No. 4,051,234, Sep. 27, 1977 to Gieske et al. Preferred antimicrobial agents include zinc salts, stannous salts, cetylpyridinium chloride, chlorhexidine, triclosan, triclosan monophosphate, and flavor oils such as thymol. Triclosan and other agents of this type are disclosed in Parran, Jr. et al., U.S. Pat. No. 5,015,466, issued May 14, 1991, and U.S. Pat. No. 4,894,220, Jan. 16, 1990 to Nabi et al. These agents provide anti-plaque benefits and are typically present at levels of from about 0.01% to about 5.0%, by weight of the composition.

Particularly for mouthrinse compositions, a preferred active is cetylpyridinium chloride (CPC), a quaternary ammonium compound with an aliphatic chain (C=16) classified as a cationic surface-active agent (The United States Pharmacopeia-23, The National Formulary 18, p. 329, 1995). As such, it has both a positively charged hydrophilic region and a hydrophobic region. CPC has been shown to possess antimicrobial activity against a number of oral bacteria (R. N. Smith, et al., “Inhibition of Intergeneric Co-aggregation Among Oral Bacteria by Cetylpyridinium Chloride, Chlorhexidine Digluconate and Octenidine Dihydrochloride,” J. of Periodontal Research, 1991, 26: 422-429). The mechanism of action of CPC is dependent upon the ability of this positively charged molecule to interact with negatively charged anionic sites on the bacterial cell walls. The cetylpyridinium chloride is included in the present compositions at levels of at least about 0.035%, typically from about 0.045% to about 1.0% or from about 0.05% to about 0.10% by weight of the composition.

Another optional active agent that may be added to the present compositions is a dentinal desensitizing agent to control hypersensitivity, such as salts of potassium, calcium, strontium and tin including nitrate, chloride, fluoride, phosphates, pyrophosphate, polyphosphate, citrate, oxalate and sulfate.

In addition to the components described above, the present compositions may comprise additional optional components collectively referred to as orally acceptable carrier materials, which are described in the following paragraphs.

### Orally Acceptable Carrier Materials

The orally acceptable carrier comprises one or more compatible solid or liquid excipients or diluents which are suitable for topical oral administration. By “compatible,” as used herein, is meant that the components of the composition are capable of being commingled without interaction in a manner which would substantially reduce the composition's stability and/or efficacy.

The carriers or excipients of the present invention can include the usual and conventional components of dentifrices, non-abrasive gels, subgingival gels, mouthwashes or rinses, mouth sprays, chewing gums, lozenges and breath mints as more fully described hereinafter.

The choice of a carrier to be used is basically determined by the way the composition is to be introduced into the oral cavity. Carrier materials for toothpaste, tooth gel or the like include abrasive materials, sudsing agents, binders, humectants, flavoring and sweetening agents, etc. as disclosed in e.g., U.S. Pat. No. 3,988,433 to Benedict. Carrier materials for biphasic dentifrice formulations are disclosed in U.S. Pat. No. 5,213,790 issued May 23, 1993; U.S. Pat. No. 5,145,666 issued Sep. 8, 1992; and U.S. Pat. No. 5,281,410 issued Jan. 25, 1994 all to Lukacovic et al. and in U.S. Pat. Nos. 4,849,213 and 4,528,180 to Schaeffer. Mouthwash, rinse or mouth spray carrier materials typically include water, flavoring and sweetening agents, etc., as disclosed in, e.g., U.S. Pat. No. 3,988,433 to Benedict. Lozenge carrier materials typically include a candy base; chewing gum carrier materials include a gum base, flavoring and sweetening agents, as in, e.g., U.S. Pat. No. 4,083,955, to Grabenstetter et al. Sachet carrier materials typically include a sachet bag, flavoring and sweetening agents. For subgingival gels used for delivery of actives into the periodontal pockets or around the periodontal pockets, a “subgingival gel carrier” is chosen as disclosed in, e.g. U.S. Pat. Nos. 5,198,220 and 5,242,910 both to Damani. Carriers suitable for the preparation of compositions of the present invention are well known in the art. Their selection will depend on secondary considerations like taste, cost, and shelf stability, etc.

The compositions of the present invention may also be in the form of non-abrasive gels and subgingival gels, which may be aqueous or non-aqueous. In still another aspect, the invention provides a dental implement impregnated with the present composition. The dental implement comprises an implement for direct contact with teeth and other tissues in the oral cavity, the implement being impregnated or coated with the present composition. The dental implement can be impregnated fibers and polymeric materials in the form of dental floss or tape, chips, strips, and films.

In a preferred embodiment, the compositions of the subject invention are in the form of dentifrices, such as toothpastes, tooth gels and tooth powders. Components of such toothpaste and tooth gels generally include in addition to the components discussed above, one or more of a dental abrasive (from about 6% to about 50%), a surfactant (from about 0.5% to about 10%), a thickening agent (from about 0.1% to about 5%), a humectant (from about 10% to about 55%), a flavoring agent (from about 0.04% to about 2%), a sweetening agent (from about 0.1% to about 3%), a coloring agent (from about 0.01% to about 0.5%) and water (from about 2% to about 45%). Such toothpaste or tooth gel may also include one or more of an anticaries agent (from about 0.05% to about 0.3% as fluoride ion) and an anticalculus agent (from about 0.1% to about 13%). Tooth powders, of course, contain substantially all non-liquid components.

Other preferred embodiments of the subject invention are liquid products, including mouthwashes or rinses, mouth sprays, dental solutions and irrigation fluids. Components of such mouthwashes and mouth sprays typically include in addition to the components discussed above, one or more of water (from about 45% to about 95%), ethanol (from about 0% to about 25%), a humectant (from about 0% to about 50%), a surfactant (from about 0.01% to about 7%), a flavoring agent (from about 0.04% to about 2%), a sweetening agent (from about 0.1% to about 3%), and a coloring agent (from about 0.001% to about 0.5%). Such mouthwashes and mouth sprays may also include one or more of an anticaries agent (from about 0.05% to about 0.3% as fluoride ion) and an anticalculus agent (from about 0.1% to about 3%). Components of dental solutions generally include one or more of water (from about 90% to about 99%), preservative (from about 0.01% to about 0.5%), thickening agent (from 0% to about 5%), flavoring agent (frorn about 0.04% to about 2%), sweetening agent (from about 0.1% to about 3%), and surfactant (from 0% to about 5%).

Types of orally acceptable carriers or excipients which may be included in compositions of the present invention, along with specific non-limiting examples, are discussed in the following paragraphs.

### Fluoride Source

It is common to have a water-soluble fluoride compound present in dentifrices and other oral compositions in an amount sufficient to give a fluoride ion concentration in the composition, and/or when it is used of from about 0.0025% to about 5.0% by weight, preferably from about 0.005% to about 2.0% by weight, to provide anticaries effectiveness. A wide variety of fluoride ion-yielding materials can be employed as sources of soluble fluoride in the present compositions. Examples of suitable fluoride ion-yielding materials are found in U.S. Pat. No. 3,535,421, Oct. 20, 1970 to Briner et al. and U.S. Pat. No. 3,678,154, Jul. 18, 1972 to Widder et al. Representative fluoride ion sources include: stannous fluoride, sodium fluoride, potassium fluoride, sodium monofluorophosphate, indium fluoride, amine fluoride and many others. Stannous fluoride and sodium fluoride are preferred, as well as mixtures thereof.

### Abrasives

Dental abrasives useful in the compositions of the subject invention include many different materials. The material selected must be one which is compatible within the composition of interest and does not excessively abrade dentin. Suitable abrasives include, for example, silicas including gels and precipitates, insoluble sodium polymetaphosphate, hydrated alumina, calcium carbonate, dicalcium orthophosphate dihydrate, calcium pyrophosphate, tricalcium phosphate, calcium polymetaphosphate, and resinous abrasive materials such as particulate condensation products of urea and formaldehyde.

Another class of abrasives for use in the present compositions is the particulate thermo-setting polymerized resins as described in U.S. Pat. No. 3,070,510 issued to Cooley & Grabenstetter on Dec. 25, 1962. Suitable resins include, for example, melamines, phenolics, ureas, melamine-ureas, melamine-formaldehydes, urea-formaldehyde, melamine-urea-formaldehydes, cross-linked epoxides, and cross-linked polyesters.

Silica dental abrasives of various types are preferred because of their unique benefits of exceptional dental cleaning and polishing performance without unduly abrading tooth enamel or dentine. The silica abrasive polishing materials herein, as well as other abrasives, generally have an average particle size ranging between about 0.1 to about 30 microns, and preferably from about 5 to about 15 microns. The abrasive can be precipitated silica or silica gels such as the silica xerogels described in Pader et al., U.S. Pat. No. 3,538,230, issued Mar. 2, 1970, and DiGiulio, U.S. Pat. No. 3,862,307, issued Jan. 21, 1975. Examples include the silica xerogels marketed under the trade name “Syloid” by the W. R. Grace & Company, Davison Chemical Division and precipitated silica materials such as those marketed by the J. M. Huber Corporation under the trade name, Zeodent®, particularly the silicas carrying the designation Zeodent® 119, Zeodent® 118, Zeodent® 109 and Zeodent® 129. The types of silica dental abrasives useful in the toothpastes of the present invention are described in more detail in Wason, U.S. Pat. No. 4,340,583 issued Jul. 29, 1982; and in commonly-assigned U.S. Pat. No. 5,603,920 issued on Feb. 18, 1997; U.S. Pat. No. 5,589,160 issued Dec. 31, 1996; U.S. Pat. No. 5,658,553 issued Aug. 19, 1997; U.S. Pat. No. 5,651,958 issued Jul. 29, 1997, and U.S. Pat. No. 6,740,311 issued May 25, 2004.

Mixtures of abrasives can be used such as mixtures of the various grades of Zeodent® silica abrasives listed above. The total amount of abrasive in dentifrice compositions of the subject invention typically range from about 6% to about 70% by weight; toothpastes preferably contain from about 10% to about 50% of abrasives, by weight of the composition. Dental solution, mouth spray, mouthwash and non-abrasive gel compositions of the subject invention typically contain little or no abrasive.

### Anticalculus Agent

The present compositions may optionally include an additional anticalculus agent, such as a pyrophosphate salt as a source of pyrophosphate ion. The pyrophosphate salts useful in the present compositions include the dialkali metal pyrophosphate salts, tetraalkali metal pyrophosphate salts, and mixtures thereof. Disodium dihydrogen pyrophosphate (Na₂H₂P₂O₇), tetrasodium pyrophosphate (Na₄P₂O₇), and tetrapotassium pyrophosphate (K₄P₂O₇) in their unhydrated as well as hydrated forms are the preferred species. In compositions of the present invention, the pyrophosphate salt may be present in one of three ways: predominately dissolved, predominately undissolved, or a mixture of dissolved and undissolved pyrophosphate.

Compositions comprising predominately dissolved pyrophosphate refer to compositions where at least one pyrophosphate ion source is in an amount sufficient to provide at least about 1.0% free pyrophosphate ions. The amount of free pyrophosphate ions may be from about 1% to about 15%, from about 1.5% to about 10% in one embodiment, and from about 2% to about 6% in another embodiment. Free pyrophosphate ions may be present in a variety of protonated states depending on the pH of the composition.

Compositions comprising predominately undissolved pyrophosphate refer to compositions containing no more than about 20% of the total pyrophosphate salt dissolved in the composition, preferably less than about 10% of the total pyrophosphate dissolved in the composition. Tetrasodium pyrophosphate salt is a preferred pyrophosphate salt in these compositions. Tetrasodium pyrophosphate may be the anhydrous salt form or the decahydrate form, or any other species stable in solid form in the dentifrice compositions. The salt is in its solid particle form, which may be its crystalline and/or amorphous state, with the particle size of the salt preferably being small enough to be aesthetically acceptable and readily soluble during use. The amount of pyrophosphate salt useful in making these compositions is any tartar control effective amount, generally from about 1.5% to about 15%, preferably from about 2% to about 10%, and most preferably from about 3% to about 8%, by weight of the dentifrice composition.

Compositions may also comprise a mixture of dissolved and undissolved pyrophosphate salts. Any of the above mentioned pyrophosphate salts may be used.

The pyrophosphate salts are described in more detail in Kirk-Othmer Encyclopedia of Chemical Technology, Third Edition, Volume 17, Wiley-Interscience Publishers (1982).

Optional agents to be used in place of or in combination with the pyrophosphate salt include such known materials as synthetic anionic polymers, including polyacrylates and copolymers of maleic anhydride or acid and methyl vinyl ether (e.g., Gantrez), as described, for example, in U.S. Pat. No. 4,627,977, to Gaffar et al., as well as, e.g., polyamino propane sulfonic acid (AMPS), diphosphonates (e.g., EHDP; AHP), polypeptides (such as polyaspartic and polyglutamic acids), and mixtures thereof.

### Chelating Agents

Another optional agent is a chelating agent, also called sequestrants, such as gluconic acid, tartaric acid, citric acid and pharmaceutically-acceptable salts thereof. Chelating agents are able to complex calcium found in the cell walls of the bacteria. Chelating agents can also disrupt plaque by removing calcium from the calcium bridges which help hold this biomass intact. However, it is not desired to use a chelating agent which has an affinity for calcium that is too high, as this may result in tooth demineralization, which is contrary to the objects and intentions of the present invention. Suitable chelating agents will generally have a calcium binding constant of about 10¹ to 10⁵ to provide improved cleaning with reduced plaque and calculus formation. Chelating agents also have the ability to complex with metallic ions and thus aid in preventing their adverse effects on the stability or appearance of products. Chelation of ions, such as iron or copper, helps retard oxidative deterioration of finished products.

Examples of suitable chelating agents are sodium or potassium gluconate and citrate; citric acid/alkali metal citrate combination; disodium tartrate; dipotassium tartrate; sodium potassium tartrate; sodium hydrogen tartrate; potassium hydrogen tartrate; sodium, potassium or ammonium polyphosphates and mixtures thereof. The amounts of chelating agent suitable for use in the present invention are about 0.1% to about 2.5%, preferably from about 0.5% to about 2.5% and more preferably from about 1.0% to about 2.5%.

Still other chelating agents suitable for use in the present invention are the anionic polymeric polycarboxylates. Such materials are well known in the art, being employed in the form of their free acids or partially or preferably fully neutralized water soluble alkali metal (e.g. potassium and preferably sodium) or ammonium salts. Examples are 1:4 to 4:1 copolymers of maleic anhydride or acid with another polymerizable ethylenically unsaturated monomer, preferably methyl vinyl ether (methoxyethylene) having a molecular weight (M.W.) of about 30,000 to about 1,000,000. These copolymers are available for example as Gantrez AN 139 (M.W. 500,000), AN 119 (M.W. 250,000) and S-97 Pharmaceutical Grade (M.W. 70,000), of GAF Chemicals Corporation.

Other operative polymeric polycarboxylates include the 1:1 copolymers of maleic anhydride with ethyl acrylate, hydroxyethyl methacrylate, N-vinyl-2-pyrrolidone, or ethylene, the latter being available for example as Monsanto EMA No. 1103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone.

Additional operative polymeric polycarboxylates are disclosed in U.S. Pat. No. 4,138,477, Feb. 6, 1979 to Gaffar and U.S. Pat. No. 4,183,914, Jan. 15, 1980 to Gaffar et al. and include copolymers of maleic anhydride with styrene, isobutylene or ethyl vinyl ether; polyacrylic, polyitaconic and polymaleic acids; and sulfoacrylic oligomers of M.W. as low as 1,000 available as Uniroyal ND-2.

### Surfactants

The present compositions may also comprise surfactants, also commonly referred to as sudsing agents. Suitable surfactants are those which are reasonably stable and foam throughout a wide pH range. The surfactant may be anionic, nonionic, amphoteric, zwitterionic, cationic, or mixtures thereof.

Anionic surfactants useful herein include the water-soluble salts of alkyl sulfates having from 8 to 20 carbon atoms in the alkyl radical (e.g., sodium alkyl sulfate) and the water-soluble salts of sulfonated monoglycerides of fatty acids having from 8 to 20 carbon atoms. Sodium lauryl sulfate (SLS) and sodium coconut monoglyceride sulfonates are examples of anionic surfactants of this type. Other suitable anionic surfactants are sarcosinates, such as sodium lauroyl sarcosinate, taurates, sodium lauryl sulfoacetate, sodium lauroyl isethionate, sodium laureth carboxylate, and sodium dodecyl benzenesulfonate. Mixtures of anionic surfactants can also be employed. Many suitable anionic surfactants are disclosed by Agricola et al., U.S. Pat. No. 3,959,458, issued May 25, 1976. The present composition typically comprises an anionic surfactant at a level of from about 0.025% to about 9%, from about 0.05% to about 5% in some embodiments, and from about 0.1% to about 1% in other embodiments.

Another suitable surfactant is one selected from the group consisting of sarcosinate surfactants, isethionate surfactants and taurate surfactants. Preferred for use herein are alkali metal or ammonium salts of these surfactants, such as the sodium and potassium salts of the following: lauroyl sarcosinate, myristoyl sarcosinate, palmitoyl sarcosinate, stearoyl sarcosinate and oleoyl sarcosinate. The sarcosinate surfactant may be present in the compositions of the present invention from about 0.1% to about 2.5%, preferably from about 0.5% to about 2.0% by weight of the total composition.

Cationic surfactants useful in the present invention include derivatives of aliphatic quaternary ammonium compounds having one long alkyl chain containing from about 8 to 18 carbon atoms such as lauryl trimethylammonium chloride; cetyl pyridinium chloride; cetyl trimethylammonium bromide; di-isobutylphenoxyethyl-dimethylbenzylammonium chloride; coconut alkyltrimethylammonium nitrite; cetyl pyridinium fluoride; etc. Preferred compounds are the quaternary ammonium fluorides described in U.S. Pat. No. 3,535,421, Oct. 20, 1970, to Briner et al., where said quaternary ammonium fluorides have detergent properties. Certain cationic surfactants can also act as germicides in the compositions disclosed herein. Cationic surfactants such as chlorhexidine, although suitable for use in the current invention, are not preferred due to their capacity to stain the oral cavity's hard tissues. Persons skilled in the art are aware of this possibility and should incorporate cationic surfactants only with this limitation in mind.

Nonionic surfactants that can be used in the compositions of the present invention include compounds produced by the condensation of alkylene oxide groups (hydrophilic in nature) with an organic hydrophobic compound which may be aliphatic or alkylaromatic in nature. Examples of suitable nonionic surfactants include the Pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures of such materials.

Zwitterionic synthetic surfactants useful in the present invention include derivatives of aliphatic quaternary ammonium, phosphonium, and sulfonium compounds, in which the aliphatic radicals can be straight chain or branched, and wherein one of the aliphatic substituents contains from about 8 to 18 carbon atoms and one contains an anionic water-solubilizing group, e.g., carboxy, sulfonate, sulfate, phosphate or phosphonate.

Suitable betaine surfactants are disclosed in U.S. Pat. No. 5,180,577 to Polefka et al., issued Jan. 19, 1993. Typical alkyl dimethyl betaines include decyl betaine or 2-(N-decyl-N,N-dimethylammonio) acetate, coco betaine or 2-(N-coc-N, N-dimethyl ammonio) acetate, myristyl betaine, palmityl betaine, lauryl betaine, cetyl betaine, cetyl betaine, stearyl betaine, etc. The amidobetaines are exemplified by cocoamidoethyl betaine, cocoamidopropyl betaine, lauramidopropyl betaine and the like. The betaines of choice are preferably the cocoamidopropyl betaine and, more preferably, the lauramidopropyl betaine.

### Thickening Agents

In preparing toothpaste or gels, thickening agents are added to provide a desirable consistency to the composition, to provide desirable active release characteristics upon use, to provide shelf stability, and to provide stability of the composition, etc. Suitable thickening agents include one or a combination of carboxyvinyl polymers, carrageenan, hydroxyethyl cellulose (HEC), natural and synthetic clays (e.g., Veegum and laponite) and water soluble salts of cellulose ethers such as sodium carboxymethylcellulose (CMC) and sodium carboxymethyl hydroxyethyl cellulose. Natural gums such as gum karaya, xanthan gum, gum arabic, and gum tragacanth can also be used. Colloidal magnesium aluminum silicate or finely divided silica can be used as part of the thickening agent to further improve texture.

Suitable carboxyvinyl polymers useful as thickening or gelling agents include carbomers which are homopolymers of acrylic acid crosslinked with an alkyl ether of pentaerythritol or an alkyl ether of sucrose. Carbomers are commercially available from B.F. Goodrich as the Carbopol® series, including Carbopol 934, 940, 941, 956, and mixtures thereof.

Thickening agents are typically present in an amount from about 0.1% to about 15%, preferably from about 2% to about 10%, more preferably from about 4% to about 8%, by weight of the total toothpaste or gel composition, can be used. Higher concentrations may be used for chewing gums, lozenges and breath mints, sachets, non-abrasive gels and subgingival gels.

### Humectants

Another optional carrier material of the present compositions is a humectant. The humectant serves to keep toothpaste compositions from hardening upon exposure to air, to give compositions a moist feel to the mouth, and, for particular humectants, to impart desirable sweetness of flavor to toothpaste compositions. The humectant, on a pure humectant basis, generally comprises from about 0% to about 70%, preferably from about 5% to about 25%, by weight of the compositions herein. Suitable humectants for use in compositions of the subject invention include edible polyhydric alcohols such as glycerin, sorbitol, xylitol, butylene glycol, polyethylene glycol, propylene glycol and trimethyl glycine.

### Miscellaneous Carrier Materials

Water employed in the preparation of commercially suitable oral compositions should preferably be of low ion content and free of organic impurities. Water generally comprises up to about 99% by weight of the aqueous compositions herein. These amounts of water include the free water which is added plus that which is introduced with other materials, such as with sorbitol.

The present invention may also include an alkali metal bicarbonate salt, which may serve a number of functions including abrasive, deodorant, buffering and adjusting pH. Alkali metal bicarbonate salts are soluble in water and unless stabilized, tend to release carbon dioxide in an aqueous system. Sodium bicarbonate, also known as baking soda, is a commonly used alkali metal bicarbonate salt. The present composition may contain from about 0.5% to about 30%, preferably from about 0.5% to about 15%, and most preferably from about 0.5% to about 5% of an alkali metal bicarbonate salt.

The pH of the present compositions may be adjusted through the use of buffering agents. Buffering agents, as used herein, refer to agents that can be used to adjust the pH of aqueous compositions such as mouthrinses and dental solutions preferably to a range of about pH 4.0 to about pH 6.0 for peroxide stability. Buffering agents include sodium bicarbonate, monosodium phosphate, trisodium phosphate, sodium hydroxide, sodium carbonate, sodium acid pyrophosphate, citric acid, and sodium citrate. Buffering agents are typically included at a level of from about 0.5% to about 10%, by weight of the present compositions.

Poloxamers may be employed in the present compositions. A poloxamer is classified as a nonionic surfactant and may also function as an emulsifying agent, binder, stabilizer, and other related functions. Poloxamers are difunctional block-polymers terminating in primary hydroxyl groups with molecular weights ranging from 1,000 to above 15,000. Poloxamers are sold under the tradename of Pluronics and Pluraflo by BASF. Suitable poloxamers for this invention are Poloxamer 407 and Pluraflo L4370.

Other emulsifying agents that may be used in the present compositions include polymeric emulsifiers such as the Pemulen® series available from B.F. Goodrich, and which are predominantly high molecular weight polyacrylic acid polymers useful as emulsifiers for hydrophobic substances.

Titanium dioxide may also be added to the present composition. Titanium dioxide is a white powder which adds opacity to dentifrice compositions. Titanium dioxide generally comprises from about 0.25% to about 5% by weight of compositions.

Other optional agents that may be used in the present compositions include dimethicone copolyols selected from alkyl- and alkoxy-dimethicone copolyols, such as C12 to C20 alkyl dimethicone copolyols and mixtures thereof. Highly preferred is cetyl dimethicone copolyol marketed under the trade name Abil EM90. The dimethicone copolyol is generally present in a level of from about 0.01% to about 25%, preferably from about 0.1% to about 5%, more preferably from about 0.5% to about 1.5% by weight. The dimethicone copolyols aid in providing positive tooth feel benefits.

### Who Method of Use

The present invention also relates to methods of treating the oral cavity by use of the stable peroxide containing compositions, such as for treating and preventing plaque, gingivitis, and oral malodor, for whitening teeth and preventing staining. The benefits of these compositions may increase over time when the composition is used repeatedly.

The method of treatment herein comprises contacting a subject's dental enamel surfaces and mucosa in the mouth with the oral compositions according to the present invention. The method of treatment may be by brushing with a dentifrice or rinsing with a dentifrice slurry or mouthrinse. Other methods include contacting the topical oral gel, denture product, mouthspray, or other form with the subject's teeth and oral mucosa. The subject may be any person or animal whose tooth surface contact the oral composition. By animal is meant to include household pets or other domestic animals, or animals kept in captivity.

For example, a method of treatment may include a person brushing a dog's teeth with one of the dentifrice compositions. Another example would include the rinsing of a cat's mouth with an oral composition for a sufficient amount of time to see a benefit. Pet care products such as chews and toys may be formulated to contain the present oral compositions. The composition is incorporated into a relatively supple but strong and durable material such as rawhide, ropes made from natural or synthetic fibers, and polymeric articles made from nylon, polyester or thermoplastic polyurethane. As the animal chews, licks or gnaws the product, the incorporated active elements are released into the animal's oral cavity into a salivary medium, comparable to an effective brushing or rinsing.

### EXAMPLES

The following examples further describe and demonstrate embodiments within the scope of the present invention. These examples are given solely for the purpose of illustration and are not to be construed as limitations of the present invention as many variations thereof are possible without departing from the spirit and scope.

### Example I

### Mouthrinse Compositions

Mouthrinse compositions IA-ID are prepared by mixing the following ingredients shown in % by weight of the composition. Formulation IA includes Glass H as radical scavenger. Formulations IB to ID are treated with polymer supported chelant to reduce the trace metals that result in the formation of free radicals. Formulation IC contains stannous chloride as radical scavenger; formulation ID contains stannous chloride and propyl gallate as radical scavengers.

| Ingredients | IA | IB | IC | ID |
|---|---|---|---|---|
| Water | 84.62 | 61.83 | 85.32 | 61.51 |
| Poloxamer 407 | 0.750 | 0.70 | 0.750 | 0.70 |
| Glycerin | | 20.0 | | 20.0 |
| Propylene Glycol | | 4.0 | | 4.0 |
| Glass H Polyphosphate | 1.0 | | | |
| Stannous Chloride | | | 0.30 | 0.30 |
| Propyl Gallate | | | | 0.02 |
| Cosmetic Peroxide 35% | 4.28 | 4.28 | 4.28 | 4.28 |
| Cetylpyridinium Chloride | | 0.10 | | 0.10 |
| Sodium Saccharin | 0.06 | | 0.06 | |
| Sucralose | | 0.05 | | 0.05 |
| Sodium Citrate | 0.21 | | 0.21 | |
| Citric acid | 0.05 | | 0.05 | |
| Flavor | 0.05 | 0.05 | 0.05 | 0.05 |
| Alcohol | 8.97 | 8.97 | 8.97 | 8.97 |

### Example II

### Stability of Compositions

The stability of the present compositions was assessed by measuring any changes in levels of peroxide, active component (cetylpyridinium chloride) and flavor components under storage conditions at 40° C. and 75% Relative Humidity.

Hydrogen peroxide was measured using an aqueous compatible PeroXOquant™ quantitative peroxide assay which detects peroxide based on the oxidation of ferrous to ferric ion in the presence of xylenol orange. Peroxide first reacts with sorbitol, converting it to a peroxyl radical, which in turn initiates the Fe²⁺ oxidation to Fe³⁺. The Fe³⁺ complexes with the xylenol orange dye to produce a purple product. This complex is measured using a microplate spectrophotometer at a wavelength of 595 nm to determine the hydrogen peroxide in the sample. The method has a margin of error up to about 10%.

### Effect of Metal Removal

Two batches of formulation IB were prepared. One batch was treated with polymer supported chelant to reduce trace metals that can result in the formation of free radicals. The other batch was not treated. Treatment resulted in overall decrease in metal content and metal-mediated radical activity in the composition. The metal concentrations (ug/ml, ppb) in the untreated and treated samples are reported in Table 1 below. Metal analysis was carried out by high resolution ICP-MS at Elemental Analysis Inc, Lexington, Ky. The amounts of some metals in the samples were less than the limit of detection (LOD) of the method and are reported below as less than such LOD.

TABLE 1
Metal Analysis of Treated and Untreated Samples

| | Metal | | | | | | | | | |
|---|---|---|---|---|---|---|---|---|---|---|
| | Cr | Mn | Fe | Co | Ni | Cu | Mo | Pd | Ag | Pt |
| Untreated | 2.75 | 0.29 | 15.5 | <0.051 | <2.7 | 1.52 | 0.364 | <0.076 | <0.048 | <0.033 |
| Treated | 1.63 | 0.49 | 5.89 | <0.050 | <2.7 | 0.64 | 0.163 | <0.075 | <0.048 | <0.033 |

A flavor mix consisting of Ethylbutyrate, Limonene, Decanal, Methylsalicylate, Carvone and Anethole (each flavor component at 0.005%) was added to the compositions. The resulting mouthrinse formulations were packaged in 500 ml PET bottles and placed on accelerated stability test chamber at 40° C. and 75% Relative Humidity conditions. The amount of peroxide in the samples was measured using the method described above and no significant changes were detected. However, radical activity was greater in the untreated sample vs. the treated sample as evidenced in greater degradation of flavor components in the untreated sample. The amount of each flavor component at time 0, 17 and 31 days was evaluated by gas chromatography (GC). Results are summarized in Table 2 below showing % reduction of each component and total flavor at days 17 and 31 from day 0. Treatment of the composition to reduce metal levels resulted in greater stability of the flavor as evidenced by a decrease in the amount of certain flavor components lost to decomposition. Some flavor components, e.g., methyl salicylate, appear to be relatively stable in the presence of peroxide, while others such as decanal, limonene and anethole undergo significant decomposition and would be totally lost if the composition were not treated to remove metals. The treatment also stabilized the composition by inhibiting peroxide loss.

TABLE 2

| | % Reduction | | % Reduction | |
|---|---|---|---|---|
| | at day 17 | | at day 31 | |
| Component | Treated | Untreated | Treated | Untreated |
| Ethylbutyrate | 25 | 20 | 25 | 20 |
| Limonene | 75 | 100 | 75 | 100 |
| Decanal | 25 | 80 | 75 | 80 |
| Methylsalicylate | 0 | 0 | 0 | 0 |
| Carvone | 20 | 20 | 20 | 20 |
| Anethole | 60 | 100 | 80 | 100 |
| Total Wt % Flavor Reduction | 33.33 | 51.72 | 40.74 | 55.17 |

### Effect of Radical Scavenger

Formulation IA with Glass H polyphosphate as radical scavenger was compared with a sample of formulation IB (no radical scavenger) that was not treated to remove metals. The formulations had a flavor mix consisting of Ethylbutyrate, Limonene, Decanal, Methylsalicylate, Carvone and Anethole (each flavor component at 0.005%). The resulting mouthrinse formulations were packaged in 500 ml PET bottles and placed on accelerated stability test chamber at 40° C. and 75% Relative Humidity. The amount of each flavor component at time 0, 17 and 31 days were measured by gas chromatography (GC). Results are summarized in Table 3 below, demonstrating that addition of polyphosphate radical scavenger resulted in increased stability of certain flavor components such as ethyl butyrate, limonene, decanal and anethole. Thus combining both methods, adding radical scavengers and treatment to remove metals will result in greatly enhanced formulation stability.

TABLE 3

| | % Reduction | | % Reduction | |
|---|---|---|---|---|
| | at day 17 | | at day 31 | |
| | Formula | Formula | Formula | Formula |
| Component | IA | IB | IA | IB |
| Ethyl butyrate | 0 | 20 | 0 | 20 |
| Limonene | 25 | 100 | 25 | 100 |
| Decanal | 20 | 80 | 40 | 80 |
| Methylsalicylate | 25 | 0 | 25 | 0 |
| Carvone | 50 | 20 | 50 | 20 |
| Anethole | 60 | 100 | 80 | 100 |
| Total Wt % Flavor Reduction | 33.33 | 51.72 | 43.3 | 55.17 |

### Stability of Cetylpyridinium Chloride (CPC)

Two batches of formulation IB rinse were prepared having a 0.10% initial concentration of cetylpyridinium chloride (CPC) as antimicrobial active. One batch was treated with polymer supported chelant to reduce trace metals that can result in the formation of free radicals. The other batch was not treated. The resulting mouthrinse formulations were packaged in 500 ml PET bottles and placed on accelerated stability test chamber at 40° C. and 75% Relative Humidity. The CPC concentration was measured by High Performance liquid chromatography with UV detection. Results are reported in Table 4 below demonstrating that the reduction in transition metal impurities prevented the degradation of CPC.

TABLE 4
CPC Concentration, ppm

| | Days | | | | | % Decrease in |
|---|---|---|---|---|---|---|
| | 0 | 17 | 31 | 60 | 90 | CPC Amount |
| Treated | 1028 | 1047 | 1048 | 1041 | 1041 | no change |
| Formulation IB | | | | | | |
| Untreated | 1023 | 1012 | 1006 | 987 | 978 | 4.4% |
| Formulation IB | | | | | | |

A detailed investigation of the degradation products was carried out by LC-MS-MS techniques (liquid chromatography-mass spectroscopy) and the pathway for the degradation of CPC was found to be mediated by hydroxyl radicals as illustrated below.

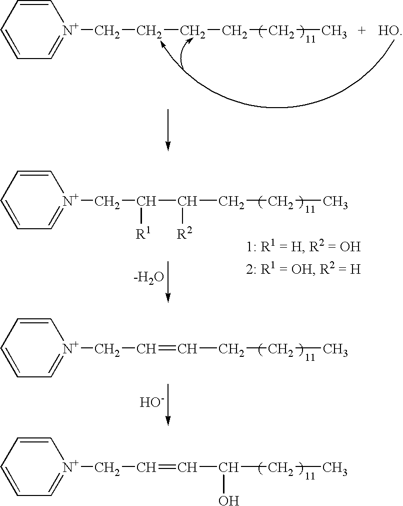

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. An oral care composition comprising
(a) from about 0.01% to about 30% by weight of the composition of a peroxide source,
(b) a flavor system comprising one or a mixture of flavorants, coolants and sweeteners, and
(c) an orally-acceptable carrier,
wherein the composition is essentially free of metals having free radical forming potential and wherein the composition is stabilized against significant loss of components through degradation mediated by free radicals.

2. An oral care composition according to claim 1 wherein the peroxide source is selected from the group consisting peroxides, perborates, percarbonates, peroxyacids, persulfates, and mixtures thereof.

3. An oral care composition according to claim 1 wherein the peroxide source is selected from the group consisting of hydrogen peroxide, urea peroxide, calcium peroxide, sodium percarbonate, and mixtures thereof.

4. An oral care composition according to claim 1 wherein the concentration in the composition of metals with radical forming potential is no more than 1.8 ppb Chromium (Cr), 0.6 ppb Manganese (Mn), 9 ppb Iron (Fe), 0.07 ppb Cobalt (Co), 10 ppb Nickel (Ni), 1 ppb Copper (Cu), 0.3 ppb Molybdnenum (Mo), 0.09 ppb Palladium (Pd), 0.06 ppb Silver (Ag), and 0.045 ppb Platinum (Pt).

5. An oral care composition according to claim 1, wherein the flavor system comprises from about 0.001% to about 5%, by weight of the composition of one or a mixture of natural or synthetic flavorants.

6. An oral care composition according to claim 1 further comprising a free radical scavenging or quenching agent.

7. An oral care composition according to claim 6, wherein the free radical scavenging or quenching agent is selected from phosphate and polyphosphate compounds, tin compounds, mono- and polyhydroxy benzenes and derivatives thereof, alkyl- and aryl carboxylates, and mixtures thereof.

8. An oral care composition according to claim 7, wherein the free radical scavenging or quenching agent comprises a phosphate compound selected from a polyphosphate containing an average number of phosphate units of from about 2 to about 125, a polyphosphorylated inositol compound, and an alkali metal, alkaline earth metal or ammonium salt thereof.

9. An oral care composition according to claim 8, wherein the free radical scavenging or quenching agent comprises a polyphosphate containing an average number of phosphate units of from about 3 to about 21.

10. An oral care composition according to claim 6, wherein the free radical scavenging or quenching agent comprises one or a mixture of propyl gallate, catechin, gallocatechin gallate, epicatechin (EC), epigallocatechin (EGC), epigallocatechin gallate (EGCG), epicatechin gallate (ECG) and proanthocyanidins.

11. An oral care composition according to claim 6, wherein the free radical scavenging or quenching agent comprises stannous chloride.

12. An oral care composition according to claim 1, further comprising an antimicrobial active selected from cetyl pyridinium chloride, domiphen bromide, zinc salts, stannous salts, chlorhexidine, triclosan, triclosan monophosphate and mixtures thereof.

13. An oral care composition according to claim 1, wherein the flavor system comprises one or a mixture of coolants selected from menthol, menthyl esters, carboxamides, ketals, and diols.

14. An oral care composition according to claim 1 in a form selected from toothpaste, dentifrice, subgingival gel, mouthrinse, mouthspray, mousse, foam and whitening gel.

15. An oral care composition comprising
(a) from about 0.01% to about 30% by weight of the composition of a peroxide source,
(b) from about 0.035% to about 1.0% by weight of a quaternary ammonium antimicrobial agent,
(b) a flavor system comprising one or a mixture of flavorants, coolants and sweeteners, and
(c) an orally-acceptable carrier,
wherein the composition is essentially free of metals having free radical forming potential and wherein the composition is stabilized against significant loss of peroxide, quaternary ammonium agent and other components through decomposition mediated by free radicals.

16. An oral care composition according to claim 15, wherein the quaternary ammonium agent comprises cetylpyridinium chloride and the peroxide source comprises hydrogen peroxide or urea peroxide.

17. A method of stabilizing peroxide-containing compositions against degradation of peroxide and other components, comprising one or a combination of
(a) treating the composition to reduce concentration of metals with free radical forming potential thereby reducing free radical activity, and
(b) incorporating an effective amount of free radical scavenger or quencher in the composition.

18. A method according to claim 17 wherein the content of metals in the composition is reduced to no more than 1.8 ppb Chromium (Cr), 0.6 ppb Manganese (Mn), 9 ppb Iron (Fe), 0.07 ppb Cobalt (Co), 10 ppb Nickel (Ni), 1 ppb Copper (Cu), 0.3 ppb Molybdnenum (Mo), 0.09 ppb Palladium (Pd), 0.06 ppb Silver (Ag), and 0.045 ppb Platinum (Pt).
